(19) United States
(12) Patent Application Publication
(10) Pub. No.: US 2010/0137300 A1
(43) Pub. Date: Jun. 3, 2010

(54) Indolizine Acetic Acid Derivatives as CRTH2 Antagonists

(76) Inventors: George Hynd, Essex (GB); Nicholas Charles Ray, Essex (GB); Harry Finch, Essex (GB); John Gary Montana, Essex (GB); Michael Colin Cramp, Essex (GB); Trevor Keith Harrison, Essex (GB); Rosa Arienzo, Essex (GB); Paul Blaney, Essex (GB); Yann Griffon, Essex (GB); David Middlemiss, Hertfordshire (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/530,929

(22) PCT Filed: Mar. 21, 2007
(86) PCT No.: PCT/GB07/00996

§ 371 Date: Jan. 27, 2010

(51) Int. Cl. A61K 31/5377; A61K 31/435; A61K 31/4709; C07D 413/12; C07D 221/04; C07D 215/14; A61P 1/00; A61P 11/06; A61P 11/00; A61P 1/04

(52) U.S. Cl. 514/233.2; 514/299; 514/314; 544/127; 546/112; 546/174; 546/183

(57) ABSTRACT

The specific compounds of this list: {7-cyano-2-methyl-1-[4-(morpholine-4-sutfonyl)benzyl]indolizin-3-yl}acetic acid, {1-(3-chloro-4-ethanesulfonylbenzyl)-7-cyano-2-methylindolizin-3-yl}acetic acid, {1-[3-chloro4-(morpholine-4-sulfonyl)benzyl]-7-cyano-2-methylindolizin-3-yl}acetic acid, {1-(3-chloro-4-ethanesulfonylphenylsulfanyl)-7-cyano-2-methylindolizin-3-yl}acetic acid, {1-[3-chloro-4-(morpholine-4-sutfonyl)phenylsutfanyl]-7-cyano-2-methylindolizin-3-yl}acetic acid, {7-cyano-1-(6-fluoroquinolin-2-ylmethyl)-2-methylindolizin-3-yl}acetic acid are ligands of the CRTH2 receptor and are useful in the treatment of respiratory diseases.

This invention relates to specific indolizine compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component.

### BACKGROUND TO THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major metabolite produced by the action of cyclooxygenase on arachadonic acid by mast cells in response, to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al; Arch. Otolarynol. Head Neck Surg., 1987, 113, 179-183; Lebel et al; J. Allergy Clin. Immunol., 1988, 82, 869-877), bronchial asthma (Murray et al; N. Engl. J. Med., 1986, 315, 800-804; Liu et al; Am. Rev. Respir. Dis., 1990, 142, 126-132; Wenzel et al; J. Allergy Clin. Immunol., 1991, 87, 540-548), and urticaria (Heavey et al; J. Allergy Clin. Immunol., 1986, 78, 458-461). PGD₂ mediates it effects through two receptors, the PGD₂ (or DP) receptor (Bole et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonise the effects of PGD₂ at its receptors may have beneficial effects in a number of disease states.

The CRTH2 receptor has been shown to be expressed on cell types associated with allergic inflammation, such as basophils, eosinophils, and Th2-type immune helper cells (Hirai et al; J. Exp. Med., 2001, 193, 255-261). The CRTH2 receptor has been shown to mediate PGD₂-mediated cell migration in these cell types (Hirai et al; J. Exp. Med., 2001, 193, 255-261), and also to play a major role in neutrophil and eosinophil cell recruitment in a model of contact dermatitis (Takeshita et al; Int. Immunol., 2004, 16, 947-959). Ramatroban {(3R)-3-[(4-fluorophenyl)sulphonyl-amino]-1,2,3,4-tetrahydro-9H-carbazole-9-propanoic acid}, a dual CRTH2 and thromboxane A₂ receptor antagonist, has been shown to attenuate these responses (Sugimoto et al; J. Pharmacol. Exp. Ther., 2003, 305, 347-352; Takeshita et al; op. cit.). The potential of PGD₂ both to enhance allergic inflammation and induce an inflammatory response has been demonstrated in mice and rats. Transgenic mice over expressing PGD₂ synthase exhibit an enhanced pulmonary eosinophilia and increased levels of Th2 cytokines in response to allergen challenge (Fujitani et al; J. Immunol., 2002, 168, 443-449). In addition, exogenously administered CRTH2 agonists enhance the allergic response in sensitised mice (Spik et al; J. Immunol., 2005, 174, 3703-3708). In rats exogenously applied CRTH2 agonists cause a pulmonary eosinophilia but a DP agonist (BW 245C) or a TP agonist (1-BOP) showed no effect (Shirashi et al; J. Pharmacol. Exp Ther, 2005, 312, 954-960). These observations suggest that CRTH2 antagonists may have valuable properties for the treatment of diseases mediated by PGD₂.

Our copending application PCT/GB2006/003394 relates to CRTH2 antagonist compounds of formula (I) and salts, N-oxides, hydrates and solvates thereof:

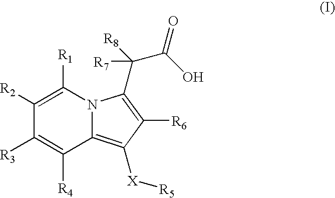

wherein
R₁, R₂, R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₁₀;
n is 0, 1 or 2;
   R₅ is C₁-C₆alkyl, fully or partially fluorinated C₁-C₆alkyl, C₁-C₆alkenyl, C₁-C₆alkynyl, optionally substituted aryl, or optionally substituted heteroaryl;
   R₆ is hydrogen, C₁-C₆alkyl or fully or partially fluorinated C₁-C₆alkyl;
   R₇ and R₈ are independently hydrogen or C₁-C₆alkyl, or R₇ and R₈ together with the atom to which they are attached form a cycloalkyl group; and
   X is —CHR₆—, —S(O)ₙ—, —NR₆SO₂— or —SO₂NR₆— wherein n is 0, 1 or 2.

### DETAILED DESCRIPTION OF THE INVENTION

The present invention provides a group of specific compounds falling within the scope of, but not specifically disclosed in our copending application PCT/GB2006/003394 referred to above.

The invention provides a compound selected from the group consisting of
{7-cyano-2-methyl-1-[4-(morpholine-4-sulfonyl)benzyl]indolizin-3-yl}acetic acid,
[1-(3-chloro-4-ethanesulfonylbenzyl)-7-cyano-2-methylindolizin-3-yl]acetic acid,
{1-[3-chloro-4-(morpholine-4-sulfonyl)benzyl]-7-cyano-2-methylindolizin-3-yl}acetic acid,
[1-(3-chloro-4-ethanesulfonylphenylsulfanyl)-7-cyano-2-methylindolizin-3-yl]acetic acid,
{1-[3-chloro-4-(morpholine-4-sulfonyl)phenylsulfanyl]-7-cyano-2-methylindolizin-3-yl}acetic acid,
[7-cyano-1-(6-fluoroquinolin-2-ylmethyl)-2-methylindolizin-3-yl]acetic acid,
and salts, N-oxides, hydrates and solvates thereof.

Compounds with which the invention is concerned are CRTH2 receptor antagonists.

A second aspect of the invention is a pharmaceutical composition comprising a compound of the invention in admixture with a pharmaceutically acceptable carrier or excipient.

A third aspect of the invention is a compound of the invention for use in therapy.

A fourth aspect of the invention is the use of a compound of the invention in the manufacture of a medicament for the treatment of a disease in which a CRTH2 antagonist can prevent, inhibit or ameliorate the pathology and/or symptomatology of the disease.

A fifth aspect of the invention is a method for treating a disease in a patient in which a CRTH2 antagonist can prevent, inhibit or ameliorate the pathology and/or symptomatology of the disease, which method comprises administering to the patient a therapeutically effective amount of a compound of the invention.

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment of asthma, chronic obstructive pulmonary, disease, rhinitis, allergic airway syndrome, or allergic rhinobronchitis. Psoriasis, atopic and non-atopic dermatitis Crohn's disease, ulcerative colitis, and irritable bowel disease are other specific conditions where the present compounds may have particular utility.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Specific salts with bases include the benzathine, calcium, diolamine, meglumine, olamine, potassium, procaine, sodium, tromethamine and zinc salts. Those compounds of the invention which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like. Where a compound contains a quaternary ammonium group acceptable counter-ions may be, for example, chlorides, bromides, sulfates, methanesulfonates, benzenesulfonates, toluenesulfonates (tosylates), napadisylates (naphthalene-1,5-disulfonates or naphthalene-1-(sulfonic acid)-5-sulfonates), edisylates (ethane-1,2-disulfonates or ethane-1-(sulfonic acid)-2-sulfonates), isethionates (2-hydroxyethylsulfonates), phosphates, acetates, citrates, lactates, tartrates, mesylates, maleates, malates, fumarates, succinates, xihafoates, p-acetamidobenzoates and the like; wherein the number of quaternary ammonium species balances the pharmaceutically acceptable salt such that the compound has no net charge.

Use of prodrugs, such as esters, of compounds with which the invention is concerned is also part of the invention. “Prodrug” means a compound which is convertible in vivo by metabolic means (e.g. by hydrolysis, reduction or oxidation) to a compound of formula (I). For example an ester prodrug of a compound of formula (I) may be convertible by hydrolysis in vivo to the parent molecule. Suitable esters of compounds of formula (I) are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluene-sulphonates, cyclohexylsulphamates and quinates. Examples of ester prodrugs are those described by F. J. Leinweber, Drug Metab. Res., 1987; 18, 379. As used in herein, references to the compounds of formula (I) are meant to also include the prodrug forms.

### Compositions

As mentioned above, the compounds with which the invention is concerned are CRTH2 receptor antagonists, and are useful in the treatment of diseases which benefit from such modulation. Examples of such diseases are referred to above, and include asthma, rhinitis, allergic airway syndrome, and allergic rhinobronchitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range will lie within the range of from about 0.001 mg to about 100 mg per kg body weight of a mammal, often 0.01 mg to about 50 mg per kg, for example 0.1 to 10 mg per kg, in single or divided doses. On the other hand, it may be necessary to use dosages outside these limits in some cases.

The compounds with which the invention is concerned may be prepared for administration by any route consistent with their pharmacokinetic properties. Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers. For delivery by inhalation, the active compound is preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active ingredient may also be administered parenterally in a sterile medium. Depending on the vehicle and concentration used, the drug can either be suspended or dissolved in the vehicle. Advantageously, adjuvants such as a local anaesthetic, Preservative and buffering agents can be dissolved in the vehicle.

Other compounds may be combined with compounds of this invention for the prevention and treatment of prostaglandin-mediated diseases. Thus the present invention is also concerned with pharmaceutical compositions, for preventing and treating PGD₂-mediated diseases comprising a therapeutically effective amount of a compound of the invention and one or more other therapeutic agents. Suitable therapeutic agents for a combination therapy with compounds of the invention include, but are not limited to: (1) corticosteroids, such as fluticaeone, budesonide or ciclesonide; (2) β2-adrenoreceptor agonists, such as salmeterol, formeterol or indacaterol; (3) leukotriene modulators, for example leukotriene antagonists such as montelukast or pranlukast or leukotriene biosynthesis inhibitors such as Zileuton or BAY-x1005; (4) anticholinergic agents, for example muscarinic-3 (M₃) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TNF-α inhibitors, for example anti-TNF monoclonal antibodies, such as Remicade and CDP-870 and TNF receptor immunoglobulin molecules, such as Enbrel; (12) inhibitors of matrix metalloprotease (MMP), for example MMP8, 9 and 12; (13) human neutrophil elastase inhibitors, such as those described in WO2005/026124 and WO2003/053930; (14) Adenosine A2a agonists such as those described in EP1052264 and EP1241176 (15) Adenosine A2b antagonists such as those described in WO2002/42298; (16) modulators of chemokine receptor function, for example antagonists of CCR3 and CCR8; (17) compounds which modulate the action of other prostanoid receptors, for example a PGD2 (DP) receptor antagonist or a thromboxane A2 antagonist; and (18) compounds which modulate Th2 function, for example, PPAR agonists.

The weight ratio of the compound of the invention to the second active ingredient may be varied and will depend upon the effective dose of each ingredient. Generally, an effective dose of each will be used.

The following examples describe the preparation of compounds of the invention:

### EXAMPLES

¹H NMR spectra were recorded at ambient temperature using a Varian Unity Inova (400 MHz) spectrometer with a triple resonance 5 mm probe spectrometer. Chemical shifts are expressed in ppm relative to tetramethylsilane. The following abbreviations have been used: br s=broad singlet, s=singlet, d=doublet, dd=double doublet, t=triplet, q=quartet, m=multiplet.

Mass Spectrometry (LCMS) experiments to determine retention times and associated mass ions were performed using the following methods:

Method A: experiments were performed on a Micromass Platform LCT spectrometer with positive ion electrospray and single wavelength UV 254 nm detection using a Higgins Clipeus C18 5 μm 100×3.0 mm column and a 2 mL/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.
Method B: experiments were performed on a Micromase Platform LCT spectrometer with positive and negative ion electrospray and ELS/Diode array detection using a Phenomenex Luna C18(2) 30×4.6 mm column and a 2 mL/minute flow rate. The solvent system was 95% solvent A and 5% solvent B for the first 0.50 minutes followed by a gradient up to 5% solvent A and 95% solvent B over the next 4 minutes. The final solvent system was held constant for a further 0.50 minutes
Method C: experiments were performed on a Agilent Scalar column C18, 5 μm (4.6×50 mm, flow rate 2.5 mL/minute) eluting with a H₂O/MeCN gradient containing 0.1% v/v formic acid over 7 minutes with UV detection at 215 and 254 nm. Gradient information: 0.0-0.1 minutes: 95% H₂O/5% MeCN; 0.1-5.0 minutes; Ramp from 95% H₂O/5% MeCN to 5% H₂O/95% MeCN; 5.0-5.5 minutes: Hold at 5% H₂O/95% MeCN; 5.5-5.6 minutes: Hold at 5% H₂O/95% MeCN, flow rate increased to 3.5 mL/minutes; 5.6-6.6 minutes: Hold at 5% H₂O/95% MeCN, flow rate 3.5 mL/minutes; 6.6-6.75 minutes: Return to 95% H₂O/5% MeCN, flow rate 3.5 mL/minutes; 6.75-6.9 minutes: Hold at 95% H₂O/5% MeCN, flow rate 3.5 mL/minutes; 6.9-7.0 minutes: Hold at 95% H₂O minutes 5% MeCN, flow rate reduced to 2.5 mL/minutes Mass spectra were, obtained using an electrospray ionization source in either the positive or negative mode.
Method D: experiments were performed on a Agilent Scalar column C18, 5 μm (4.6×50 mm, flow rate 2.5 mL/minutes) eluting with a H₂O/MeCN gradient containing 0.1% v/v NH₄OH over 7 minutes with UV detection at 215 and 254 nm. Gradient information: 0.0-0.1 min: 95% H₂O/5% MeCN; 0.1-5.0 minutes; Ramp from 95% H₂O/5% MeCN to 5% H₂O/95% MeCN; 5.0-5.5 minutes: Hold at 5% H₂O/95% MeCN; 5.5-5.6 minutes: Hold at 5% H₂O/95% MeCN, flow rate increased to 3.5 mL/minutes; 5.6-6.6 minutes: Hold at 5% H₂O/95% MeCN, flow rate 3.5 mL/minutes; 6.6-6.75 minutes: Return to 95% H₂O/5% MeCN, flow rate 3.5 mL/minutes; 6.75-6.9 minutes: Hold at 95% H₂O/5% MeCN, flow rate 3.5 mL/minutes; 6.9-7.0 minutes: Hold at 95% H₂O/5% MeCN, flow rate reduced to 2.5 mL/minutes Mass spectra were obtained using an electrospray ionization source in either the positive or negative mode.

### Example 1

### {7-cyano-2-methyl-1-[4-(morpholine-4-sulfonyl)benzyl]indolizin-3-yl}acetic acid

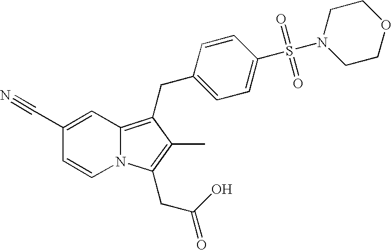

### Preparation 1a: 2-methylisonicotinonitrile

A mixture of 2-chloroisonicotinonitrile (28 g), tetrakis(triphenylphosphine)palladium(0) (5.0 g), trimethylaluminium (2.0 M in hexanes, 110 mL) and 1,4-dioxane (400 mL) was heated at reflux for 2 hours. The mixture was cooled to room temperature, diluted with 1.0 M aqueous hydrochloric acid and the organic phase extracted with 1.0 M aqueous hydrochloric acid. The combined, aqueous phases were washed with diethyl ether, basified by the addition of concentrated aqueous sodium hydroxide solution and then extracted with diethyl ether. The combined extracts were dried over magnesium sulfate and the solvent removed under reduced pressure to afford title compound, 24 g.

¹H NMR (CDCl₃): δ 2.65 (s, 3H), 7.35 (m, 1H), 7.40 (br s, 1H), 8.70 (d, J=5.0 Hz, 1H).

### Preparation 1b: 2-methylindolizine-7-carbonitrile

A mixture of 2-methylisonicotinonitrile (4.0 g), 1-bromopropan-2-one (9.3 g), sodium hydrogen carbonate (6.8 g) and acetonitrile (40 mL) was heated at reflux for 14 hours. The mixture was cooled to room temperature, diluted with water and extracted with ethyl acetate. The combined extracts were washed with saturated aqueous sodium chloride solution, dried over magnesium sulfate and the solvent removed under reduced pressure. Purification of the residue by column chromatography on silica gel, eluting with a mixture of cyclohexane and ethyl acetate gave title compound as a yellow solid, 1.5 g.

¹H NMR (CDCl₃): δ 2.35 (s, 3H), 6.50 (dd, J=1.6, 7.2 Hz, 1H), 6.55 (br s, 1H), 7.25 (br s, 1H), 7.70 (br s, 1H), 7.80 (m, 1H).

### Preparation 1c: (7-cyano-2-methylindolizin-3-yl)acetic acid ethyl ester

A solution of ethyl diazoacetate (5.4 mL) in toluene (40 mL) was added portionwise to a mixture of 2-methylindolizine-7-carbonitrile (8.1 g), copper bronze (3.3 g) and toluene (200 mL) at reflux. The resulting mixture was heated at reflux for 2 hours, cooled to room temperature and then filtered. The filtrate was concentrate under reduced pressure and the residue purified by column chromatography on silica gel, eluting with a mixture of pentane and dichloromethane (9:1 to 0:1 by volume) to afford title compound, 5.7 g.

¹H NMR (CDCl₃): δ 1.25 (t, J=7.1 Hz, 3H), 2.35 (s, 3H), 3.90 (s, 2H), 4.15 (q, J=7.1 Hz, 2H), 6.55 (m, 1H), 6.60 (d, J=1.7 Hz, 1H), 7.70 (br s, 1H), 7.85 (d, J=7.1 Hz, 1H).

### Preparation 1d: acetic acid acetoxy(4-chlorosulfonylphenyl)methyl ester

Sulfuric acid (56 g) was added dropwise to a mixture of 4-methylbenzenesulfonyl chloride (45 g), acetic acid (375 mL) and acetic anhydride (375 mL) at 0° C. and the resulting mixture was treated portionwise with chromium(VI) oxide (66 g). The mixture was stirred at room temperature for 30 minutes, poured into ice/water and the solid collected by filtration. The solid was dissolved in dichloromethane, dried over magnesium sulfate and concentrated under reduced pressure. The residue was crystallised from a mixture of acetone and hexane to afford title compound.

¹H NMR (CDCl₃): δ 2.15 (s, 6H), 7.75 (s, 1H), 7.80 (d, J=8.6 Hz, 2H), 8.10 (d, J=8.6 Hz, 2H).

### Preparation 1e: acetic acid acetoxy[4-(morpholine-4-sulfonyl)phenyl]methyl ester

A solution of morpholine (0.85 mL) in dichloromethane (5.0 mL) was added dropwise to a mixture of acetic acid acetoxy(4-chlorosulfonylphenyl)methyl ester (1.0 g) and dichloromethane (25 mL) at 0° C. and the resulting mixture was stirred at room temperature overnight. The mixture was concentrated under reduced pressure and the residue purified by column chromatography on silica gel, eluting with a mixture of cyclohexane and ethyl acetate (8:1 by volume) to afford title compound, 0.70 g.

¹H NMR (CDCl₃): δ 2.15 (s, 6H), 3.05 (m, 4H) 3.75 (m, 4H), 7.80 (s, 1H), 7.95 (d, J=8.6 Hz, 2H), 8.05 (d, J=8.6 Hz, 2H).

### Preparation 1f: 4-(morpholine-4-sulfonyl)benzaldehyde

A mixture of acetic acid acetoxy[4-(morpholine-4-sulfonyl)phenyl]methyl ester (0.70 g), water (25 mL), industrial methylated spirits (25 mL) and sodium carbonate (0.83 g) was stirred at room temperature overnight. The mixture was concentrated under reduced pressure and the residue treated with glacial acetic acid. The resulting precipitate was collected by filtration and dried to afford title compound, 0.48 g.

¹H NMR (CDCl₃): δ 3.05 (m, 4H), 3.75 (m, 4H), 7.90 (d, J=8.6 Hz, 2H), 8.10 (d, J=8.6 Hz, 2H), 10.15 (s, 1H).

### Preparation 1g: {7-cyano-2-methyl-1-[4-(morpholine-4-sulfonyl)benzyl]indolizin-3-yl}acetic acid ethyl ester

A mixture of (7-cyano-2-methylindolizin-3-yl)acetic acid ethyl ester (0.46 g), 4-(morpholine-4-sulfonyl)benzaldehyde (0.48 g) and 1,2-dichloroethane (15 mL) at 0° C. was treated dropwise with triethylsilane (1.5 mL) followed by trifluoroacetic acid (4.2 mL) and the resulting mixture was stirred at room temperature overnight. The mixture was washed with saturated aqueous sodium hydrogen carbonate solution, dried over sodium sulfate and the solvent removed under reduced pressure. The residue was purified by column chromatography on silica gel to afford title compound, 0.18 g.

¹H NMR (CDCl₃): δ 1.25 (t, J=7.3 Hz, 3H), 2.20 (s, 3H), 2.95 (m, 4H), 3.70 (m, 4H), 3.90 (s, 2H), 4.15 (q, J=7.3 Hz, 2H), 4.20 (s, 2H), 6.60 (dd, J=1.7, 7.3 Hz, 1H), 7.25 (d, J=8.5 Hz, 2H), 7.65 (d, J=8.5 Hz, 2H), 7.70 (dd, J=0.9, 1.7 Hz, 1H), 7.85 (dd, J=0.9, 7.3 Hz, 1H).

### Preparation 1h: {7-cyano-2-methyl-1-[4-(morpholine-4-sulfonyl)benzyl]indolizin-3-yl}acetic acid

A solution of {7-cyano-2-methyl-1-[4-(morpholine-4-sulfonyl)benzyl]indolizin-3-yl}acetic acid ethyl ester (0.18 g) in tetrahydrofuran (10 mL) was treated with a solution of lithium hydroxide (0.050 g) in water (10 mL) and the resulting mixture was stirred at room temperature for 1 hour. The mixture was acidified by the addition 1.0 M aqueous hydrochloric acid, extracted with ethyl acetate and the combined extracts dried over sodium sulfate. The solvent was removed under reduced pressure to afford title compound as a yellow solid, 0.059 g.

¹H NMR (CD₃OD): δ 2.15 (s, 3H), 2.85 (m, 4H), 3.60 (m, 4H), 3.95 (s, 2H), 4.25 (s, 2H), 6.60 (dd, J=1.6, 7.0 Hz, 1H), 7.35 (d, J=8.4. Hz, 2H), 7.60 (d, J=8.4 Hz, 2H), 7.90 (dd, J=0.7, 1.6 Hz, 1H), 8.00 (dd, J=0.7, 7.0 Hz, 1H).

MS: ESI (+ve) (Method A): 454 (M+H)⁺, Retention time 9.7 min.

### Example 2

### [1-(3-chloro-4-ethanesulfonylbenzyl)-7-cyano-2-methylindolizin-3-yl]acetic acid

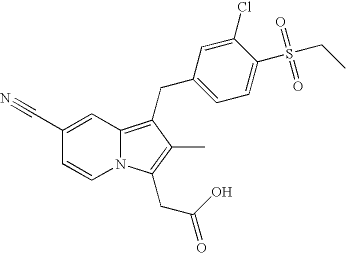

### Preparation 2a: 3-chloro-4-ethylsulfanylbenzaldehyde

A solution of 3-chloro-4-fluorobenzaldehyde (5.0 g) in N,N-dimethylformamide (10 mL) was treated with sodium ethanethiolate (2.7 g) and the resulting mixture was stirred at 70° C. for 7 hours and then at room temperature overnight. The mixture was partitioned between water (30 mL) and diethyl ether (30 mL) and the aqueous phase extracted with diethyl ether (20 mL). The combined organic phases were washed with saturated aqueous sodium chloride solution, dried over magnesium sulfate and the solvent removed under reduced pressure. Purification of the residue by column chromatography on silica gel, eluting with a mixture of iso-hexane and ethyl acetate (1:9 by volume) gave title compound as a yellow oil, 5.0 g.

### Preparation 2b: 3-chloro-4-ethanesulfonylbenzaldehyde

A solution of 3-chloro-4-ethylsulfanylbenzaldehyde (5.0 g) in dichloromethane (25 mL) at 0° C. was treated with 3-chloroperoxybenzoic acid (8.6 g) and the resulting mixture was stirred at room temperature for 2 hours. The mixture was diluted with saturated aqueous sodium hydrogen carbonate solution and the organic phase was washed with saturated aqueous sodium sulfite solution and water. The pH of the combined aqueous phases was adjusted to 6 and then extracted with dichloromethane, ethyl acetate and chloroform. The combined organic phases were dried over magnesium sulfate and the solvent removed under reduced pressure. Purification of the residue by column chromatography on silica gel, eluting with a mixture of ethyl acetate and iso-hexane (1:9 to 3:7 by volume) gave title compound, 0.60 g.

¹H NMR (CDCl₃): δ 1.30 (t, 3H), 3.50 (q, 2H), 8.0 (dd, 1H), 8.05 (d, 1H), 8.30 (d, 1H), 10.10 (s, 1H).

### Preparation 2c: [1-(3-chloro-4-ethanesulfonylbenzyl)-7-cyano-2-methylindolizin-3-yl]acetic acid ethyl ester

The title compound was prepared by the method of Preparation 1g using (7-cyano-2-methylindolizin-3-yl)acetic acid ethyl ester and 3-chloro-4-ethanesulfonylbenzaldehyde.

¹H NMR (DMSO-d6): δ 1.10 (t, 3H), 1.15 (t, 3H), 2.10 (s, 3H), 3.45 (q, 2H), 4.05 (q, 2H), 4.10 (s, 2H), 4.30 (s, 1H), 6.75 (dd, 1H), 7.35 (dd, 1H), 7.50 (d, 1H), 7.90 (d, 1H), 8.15 (d, 1H), 8.30 (s, 1H).

MS: ESI (+ve) (Method D): 459 (M+H)⁺, Retention time 4.2 min.

### Preparation 2d: [1-(3-chloro-4-ethanesulfonylbenzyl)-7-cyano-2-methylindolizin-3-yl]acetic acid

A mixture of [1-(3-chloro-4-ethanesulfonylbenzyl)-7-cyano-2-methylindolizin-3-yl]acetic acid ethyl ester (0.025 g), ethanol (0.5 mL) and tetrahydrofuran (0.3 mL) was treated with 1.0 M aqueous lithium hydroxide solution (0.14 mL) and the resulting mixture was stirred at room temperature for 1 hour. The mixture was concentrated under reduced pressure, diluted with water and the pH adjusted to 4 by the addition of glacial acetic acid. The mixture was extracted with dichloromethane and the combined extracts were dried over magnesium sulfate and then concentrated under reduced pressure to afford title compound as a green solid, 0.020 g.

¹H NMR (DMSO-d6): δ 1.10 (t, 3H), 2.10 (s, 3H), 3.45 (q, 2H), 4.00 (s, 2H), 4.25 (s, 1H), 6.75 (dd, 1H), 7.35 (dd, 1H), 7.55 (d, 1H), 7.90 (d, 1H), 8.10 (d, 1H), 8.30 (s, 1H).

MS: ESI (+ve) (Method C): 431 (M+H)⁺, Retention time 3.1 min.

### Example 3

### {1-[3-chloro-4-(morpholine-4-sulfonyl)benzyl]-7-cyano-2-methylindolizin-3-yl}acetic acid

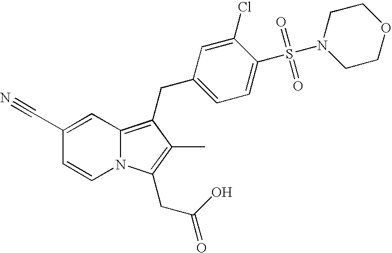

### Preparation 3a: 3-chloro-4-(morpholine-4-sulfonyl)benzonitrile

A solution of 2-chloro-4-cyanobenzenesulfonyl chloride (5.0 g) in dichloromethane (20 mL) at 0° C. was treated with morpholine (3.7 mL) and the resulting mixture was stirred at room temperature for 1 hour. The mixture was diluted with dichloromethane, washed with 10% aqueous hydrochloric acid, dilute aqueous sodium hydrogen carbonate solution and water and then dried over magnesium sulfate. The solvent was removed under reduced pressure to title compound, 5.5 g.

¹H NMR (CDCl₃): δ 3.35 (dd, 4H), 3.75 (dd, 4H), 7.70 (dd, 1H), 7.85 (d, 1H), 8.15 (d, 1H).

### Preparation 3b: 3-chloro-4-(morpholine-4-sulfonyl)benzaldehyde

A solution of 3-chloro-4-(morpholine-4-sulfonyl)benzonitrile (5.5 g) in dichloromethane (20 mL) at −75° C. was treated with diisobutylaluminium hydride (1.0 M solution in dichloromethane, 19 mL) and the resulting mixture was stirred at −78° C. for 1 hour. The mixture was treated with 1.0 M aqueous hydrochloric acid and extracted with dichloromethane. The combined extracts were washed with saturated aqueous sodium chloride solution, dried over magnesium sulfate and concentrated under reduced pressure. Purification of the residue by column chromatography on silica gel, eluting with a mixture of ethyl acetate and iso-hexane (1:9 to 1:2 by volume) gave title compound; 1.3 g.

¹H NMR (DMSO-d6): δ 3.20-3.25 (m, 4H), 3.60-3.65 (m, 4H), 8.05 (dd, 1H), 8.15-8.20 (m, 2H), 10.10 (s, 1H).

MS: ESI (+ve) (Method D): 290 (M+H)⁺, Retention time 4.0 min.

### Preparation 3c: {1-[3-chloro-4-(morpholine-4-sulfonyl)benzyl]-7-cyano-2-methylindolizin-3-yl}acetic acid ethyl ester

The title compound was prepared by the method of Preparation 1g using (7-cyano-2-methylindolizin-3-yl)acetic acid ethyl ester and 3-chloro-4-(morpholine-4-sulfonyl)benzaldehyde.

¹H NMR (DMSO-d6): δ 1.10 (t, 3H), 2.10 (s, 3H), 3.10-3.15 (m, 4H), 3.55-3.60 (m, 4H), 4.05 (q, 2H), 4.10 (s, 2H), 4.25 (s, 1H), 6.75 (dd, 1H), 7.30 (dd, 1H), 7.50 (d, 1H), 7.85 (d, 1H), 8.15 (dd, 1H), 8.30 (s, 1H).

MS: ESI (+ve) (Method D): 516 (M+H)⁺, Retention time 4.3 min.

### Preparation 3d: (1-[3-chloro-4-(morpholine-4-sulfonyl)benzyl]-7-cyano-2-methylindolizin-3-yl)acetic acid

The title compound was prepared by the method of Preparation 2d using {1-[3-chloro-4-(morpholine-4-sulfonyl)benzyl]-7-cyano-2-methylindolizin-3-yl}acetic acid ethyl ester.

¹H NMR (DMSO-d6): δ 2.10 (s, 3H), 3.10-3.15 (m, 4H), 3.55-3.60 (m, 4H), 4.00 (s, 2H), 4.25 (s, 2H), 6.75 (dd, 1H), 7.30 (dd, 1H), 7.50 (d, 1H), 7.85 (d, 1H), 8.15 (d, 1H), 8.30 (s, 1H).

MS: ESI (+ve) (Method C): 488 (M+H)⁺, Retention time 3.2 min

### Example 4

### [1-(3-chloro-4-ethanesulfonylphenylsulfanyl)-7-cyano-2-methylindolizin-3-yl]acetic acid

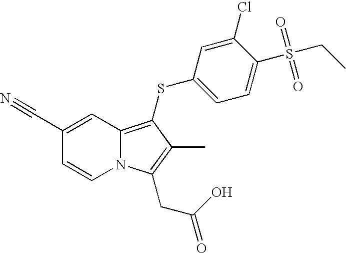

### Preparation 4a: 2-chloro-1-ethanesulfonyl-4-fluorobenzene

2-Chloro-4-fluorobenzenesulfonyl chloride (3.8 g) was added portionwise over a period of 1 hour to a solution of sodium bicarbonate (2.8 g) and sodium sulfite (4.0 g) in water (80 mL) at 75° C. and the resulting mixture was heated at 75° C. for 1 hour. The mixture was cooled to room temperature and concentrated under reduced pressure. The residue was treated with N,N-dimethylformamide (30 mL), sodium bicarbonate (2.8 g) and ethyl iodide (1.3 mL) and the resulting mixture heated at 75° C. for 2 hours and then cooled to room temperature and diluted with water (250 mL) and ethyl acetate (200 mL). The organic phase was dried over magnesium sulfate and the solvent removed under reduced pressure. Purification of the residue by column chromatography on silica gel, eluting with a mixture of ethyl acetate and iso-hexane (1:4 by volume) gave title compound, 2.1 g.

¹H NMR (CDCl₃): δ 1.25 (t, 3H), 3.40 (q, 2H), 7.20 (ddd, 1H), 7.30 (dd, 1H), 8.15 (dd, 1H).

### Preparation 4b: bis(3-chloro-4-ethanesulfonylbenzene)disulfide

A mixture of 2-chloro-1-ethanesulfonyl-4-fluorobenzene (1.8 g), sodium hydrogen sulfide (4.1 g) and 1-methylpyrrolidin-2-one (5.0 mL) was stirred at 80° C. for 1 hour and then at room temperature for 1 hour. The mixture was diluted with water, filtered and the pH of the filtrate adjusted to 1 by the addition of concentrated hydrochloric acid. The mixture was decanted and residual gum washed and decanted a further 3-times with water. The residue was partitioned between dichloromethane and water and the organic phase was washed with water, dried over magnesium sulfate and the solvent removed under reduced pressure to afford title compound, 1.4 g.

MS: ESI (+ve) (Method C): 471 (M+H)⁺, Retention time 4.2 min.

### Preparation 4c: [1-(3-chloro-4-ethanesulfonylphenylsulfanyl)-7-cyano-2-methylindolizin-3-yl]acetic acid ethyl ester

Sulfuryl chloride (0.036 mL) was added to a solution of bis(3-chloro-4-ethanesulfonylbenzene)disulfide (0.30 g) in dichloromethane (5.0 mL) at 0° C. and the resulting mixture was stirred at this temperature for 1.5 minutes and then at room temperature for 1.5 hours This mixture was then added dropwise to a solution of (7-cyano-2-methylindolizin-3-yl)acetic acid ethyl ester (0.15 g) in dichloromethane (2.0 mL) at room temperature and the resulting mixture was stirred at this temperature for 2 hours. The mixture was diluted with dichloromethane, washed with water and dried over magnesium sulfate. The solvent was removed under reduced pressure and the residue purified by column chromatography on silica gel, eluting with a mixture of ethyl acetate, diethyl ether and iso-hexane (1:4:12 by volume) to give title compound, 0.060 g.

¹H NMR (DMSO-d6): δ 1.10 (t, 3H), 1.20 (t, 3H), 2.20 (s, 3H), 3.40 (q, 2H), 4.10 (q, 2H), 4.25 (s, 2H), 7.05 (dd, 1H), 7.05 (dd, 1H), 7.20 (d, 1H), 7.80 (d, 1H), 8.10 (s, 1H), 8.45 (dd, 1H).

MS: ESI (+ve) (Method C): 477 (M+H)⁺, Retention time 4.2 min

### Preparation 4d: [1-(3-chloro-4-ethanesulfonylphenylsulfanyl)-7-cyano-2-methylindolizin-3-yl]acetic acid

The title compound was prepared by the method of Preparation 2d using [1-(3-chloro-4-ethanesulfonylphenylsulfanyl)-7-cyano-2-methylindolizin-3-yl]acetic acid ethyl ester

¹H NMR (DMSO-d6): δ 1.10 (t, 3H), 2.20 (s, 3H), 3.40 (q, 2H), 4.15 (s, 2H), 7.00-7.05 (m, 2H), 7.20 (d, 1H), 7.80 (d, 1H), 8.10 (br s, 1H), 8.40 (d, 1H), 8.45 (dd, 1H), 12.70 (br s, 1H).

MS: ESI (+ve) (Method C): 449 (M+H)⁺, Retention time 3.4 min.

### Example 5

### {1-[3-chloro-4-(morpholine-4-sulfonyl)phenylsulfanyl]-7-cyano-2-methylindolizin-3-yl}acetic acid

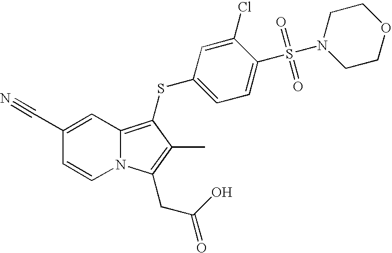

### Preparation 5a: 4-(2-chloro-4-fluorobenzenesulfonyl)morpholine

A solution of 2-chloro-4-fluorobenzenesulfonyl chloride (2.0 g) in dichloromethane (20 mL) at 0° C. was treated with morpholine (1.3 mL) and the resulting mixture was stirred at room temperature for 1 hour. The mixture was diluted with dichloromethane, washed with 10% aqueous hydrochloric acid, dilute aqueous sodium hydrogen carbonate solution and water and then dried over magnesium sulfate. The solvent was removed under reduced pressure to afford title compound, 2.3 g.

MS: ESI (+ve) (Method D): 280 (M+H)⁺, Retention time 5.2 min.

### Preparation 5b: bis(3-chloro-4-morpholinosulfonylbenzene)disulfide

The title compound was prepared by the method of Preparation 4b using 4-(2-chloro-4-fluorobenzenesulfonyl)morpholine.

### Preparation 5c: {1-[3-chloro-4-(morpholine-4-sulfonyl)phenylsulfanyl]-7-cyano-2-methylindolizin-3-yl}acetic acid ethyl ester

The title compound was prepared by the method of Preparation 4c using (7-cyano-2-methylindolizin-3-yl)acetic acid ethyl ester and bis(3-chloro-4-morpholinosulfonylbenzene)disulfide.

¹H NMR (DMSO-d6): δ 1.20 (t, 3H), 2.20 (s, 3H), 3.05-3.10 (m, 4H), 3.55-3.60 (m, 4H), 4.10 (q, 2H), 4.25 (s, 2H), 7.00 (dd, 1H), 7.05 (dd, 1H), 7.15 (d, 1H), 7.75 (d, 1H), 8.10 (br s, 1H), 8.40 (dd, 1H).

MS: ESI (+ve) (Method C): 534 (M+H)⁺, Retention time 4.3 min

### Preparation 5d: {1-[3-chloro-4-(morpholine-4-sulfonyl)phenylsulfanyl]-7-cyano-2-methylindolizln-3-yl}acetic acid

The title compound was prepared by the method of Preparation 2d {1-[3-chloro-4-(morpholine-4-sulfonyl)phenylsulfanyl]-7-cyano-2-methylindolizin-3-yl}acetic acid ethyl ester

¹H NMR (DMSO-d6): δ 2.20 (s, 3H), 3.05-3.10 (m, 4H), 3.55-3.60 (m, 4H), 4.15 (s, 2H), 7.00 (dd, 1H), 7.05 (dd, 1H), 7.20 (d, 1H), 7.75 (d, 1H), 8.10 (m, 1H), 8.40 (dd, 1H).

MS: ESI (+ve) (Method C): 506 (M+H)⁺, Retention time 3.5 min

### Example 6

### [7-cyano-1-(6-fluoroquinolin-2-ylmethyl)-2-methylindolizin-3-yl]acetic acid

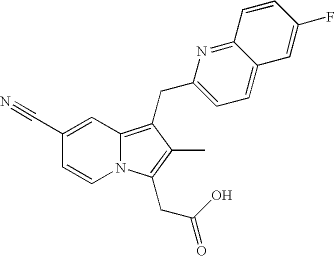

### Preparation 6a: [7-cyano-1-(6-fluoroquinolin-2-ylmethyl)-2-methylindolizin-3-yl]acetic acid ethyl ester

The title compound was prepared by the method of Preparation 1g using (7-cyano-2-methylindolizin-3-yl)acetic acid ethyl ester and 6-fluoroquinoline-2-carbaldehyde.

MS: ESI (+ve) (Method B): 402 (M+H)⁺, Retention time 3.7 min.

### Preparation 6b: [7-cyano-1-(6-fluoroquinolin-2-ylmethyl)-2-methylindolizin-3-yl]acetic acid

A solution of [7-cyano-1-(6-fluoroquinolin-2-ylmethyl)-2-methylindolizin-3-yl]acetic acid ethyl ester (0.13 g) in tetrahydrofuran (10 mL) was treated with 1.0 M aqueous lithium hydroxide solution (1.0 mL) and the resulting mixture was stirred at room temperature for 1 hour. The mixture was concentrated under reduced pressure, acidified by the addition of 1.0 M aqueous hydrochloric acid and then extracted with ethyl acetate. The combined extracts were dried over magnesium sulfate and the solvent removed under reduced pressure to afford title compound as a bright yellow solid, 0.064 g.

¹H NMR (DMSO-d6): δ 2.15 (s, 3H), 3.95 (s, 2H), 4.45 (s, 2H), 6.70 (dd, J=1.6, 7.4 Hz, 1H), 7.30 (d, J=8.4 Hz, 1H), 7.65 (dt, J=2.8, 8.8 Hz, 1H), 7.70 (dd, J=2.8, 9.4 Hz, 1H), 8.00 (dd, J=5.5, 9.4 Hz, 1H), 8.10 (d, J=7.4 Hz, 1H), 8.20 (d, J=8.4 Hz, 1H), 8.35 (s, 1H).

MS: ESI (+ve) (Method A): 374 (M+H)⁺, Retention time 9.0 min.

### Biological Methods

Compounds of the invention were tested using the following biological test methods to determine their ability to displace PGD₂ from the CRTH2 receptor and for their ability to antagonise the functional effects of PGD₂ at the CRTH2 receptor.

### Radioligand Binding Assay

The receptor binding assay is performed in a final volume of 200 μL binding buffer [10 mM BES (pH 7.4), 1 mM EDTA, 10 mM manganese chloride, 0.01% BSA] and 1 nM [³H]-PGD₂ (Amersham Biosciences UK Ltd). Ligands are added in assay buffer containing a constant amount of DMSO (1% by volume). Total binding is determined using 1% by volume of DMSO in assay buffer and non-specific binding is determined using 10 μM of unlabeled PGD₂ (Sigma). Human embryonic kidney (HEK) cell membranes (3.5 μg) expressing the CRTH2 receptor are incubated with 1.5 mg wheatgerm agglutinin SPA beads and 1 nM [³H]-PGD₂ (Amersham Biosciences UK Ltd) and the mixture incubated for 3 hours at room temperature. Bound [³H]-PGD₂ is detected using a Microbeta TRILUX liquid scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

### Functional Assay

### GTPγS Assay

The GTPγS Assay is performed in a final volume of 200 mL assay buffer (20 mM HEPES pH 7.4, 10 mM MgCl₂; 100 mM NaCl, 10 μg/mL saponin). DMSO concentrations are kept constant at 1% by volume. Human embryonic kidney (HEK) cell membranes (3.5 μg) expressing the CRTH2 receptor are incubated with the compounds for 15 min at 30° C. prior to addition of PGD₂ (30 nM final concentration) and GTP (10 μM final concentration). The assay solutions are then incubated for 30 minutes at 30° C., followed by addition of [³⁵S]-GTPγS (0.1 nM final concentration). The assay plate is than shaken and incubated for 5 minutes at 30° C. Finally, SPA beads (Amersham Biosciences, UK) are added to a final concentration of 1.5 mg/well and the plate shaken and incubated for 30 minute at 30° C. The sealed plate is centrifuged at 1000 g for 10 mins at 30 oC and the bound [³⁵S]-GTPγS is detected on Microbeta scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

### Biological Results:

The compounds of the Examples above were tested in the CRTH2 radioligand binding and GTPγS functional assays described above; the compounds all have IC₅₀ values of less than 1 μM in both assays. For example, the compound of Example 1 had an IC₅₀ value of 58 nM in the CRTH2 radioligand binding assay, and the compound of Example 6 had an IC₅₀ value of 19 nM in that assay.

1. A compound selected from the group consisting of:
{7-cyano-2-methyl-1-[4-(morpholine-4-sulfonyl)benzyl]indolizin-3-yl}acetic acid,
[1-(3-chloro-4-ethanesulfonylbenzyl)-7-cyano-2-methylindolizin-3-yl]acetic acid,
{1-[3-chloro-4-(morpholine-4-sulfonyl)benzyl]-7-cyano-2-methylindolizin-3-yl}acetic acid,
[1-(3-chloro-4-ethanesulfonylphenylsulfanyl)-7-cyano-2-methylindolizin-3-yl]acetic acid,
{1-[3-chloro-4-(morpholine-4-sulfonyl)phenylsulfanyl]-7-cyano-2-methylindolizin-3-yl}acetic acid,
[7-cyano-1-(6-fluoroquinolin-2-ylmethyl)-2-methylindolizin-3-yl]acetic acid,
and salts, N-oxides, hydrates and solvates thereof.

2. A compound as claimed in claim 1 for use in therapy.

3. A pharmaceutical composition comprising a compound as claimed in claim 1 and a pharmaceutically acceptable carrier.

4. Use of a compound as claimed in claim 1 for the manufacture of a composition for the treatment of asthma, chronic obstructive pulmonary disease, rhinitis, allergic airway syndrome, or allergic rhinobronchitis.

5. Use of a compound as claimed in claim 1 for the manufacture of a composition for the treatment of psoriasis, atopic and non-atopic dermatitis Crohn's disease, ulcerative colitis, or irritable bowel disease.

6. A method of treatment of asthma, chronic obstructive pulmonary disease, rhinitis, allergic airway syndrome, or allergic rhinobronchitis, comprising administering to a patient suffering such disease an effective amount of a compound as claimed in claim 1.

7. A method of treatment of psoriasis, atopic or non-atopic dermatitis, Crohn's disease, ulcerative colitis, or irritable bowel disease, comprising administering to a patient suffering such disease an effective amount of a compound as claimed in claim 1.
